(19) United States
(12) Patent Application Publication
(10) Pub. No.: US 2001/0033363 A1
(43) Pub. Date: Oct. 25, 2001

(54) Pair of ophthalmic lenses, range of ophthalmic lenses and method for prescribing a pair of ophthalmic lenses

(76) Inventors: Nicolas Chateau, Paris (FR); Bruno Fermigier, Paris (FR); Richard Legras, Le Plessis Robinson (FR)

Correspondence Address: YOUNG & THOMPSON, 745 SOUTH 23RD STREET 2ND FLOOR, ARLINGTON, VA, 22202

(21) Appl. No.: 09/759,461

(22) Filed: Jan. 16, 2001

(30) Foreign Application Priority Data
Jan. 14, 2000 (FR) 0000466

(51) Int. Cl. A61B 3/10

(52) U.S. Cl. 351/216

(57) ABSTRACT

The two lenses of a pair of ophthalmic lenses are of the progressive simultaneous vision type, and in one lens the progressive profile varies so that the power is greater at the center than at the periphery, and vice versa for the other lens. The range of lenses includes two series of lenses whose nominal powers differ with a predetermined increment, for example 0.25 diopter. The method of prescribing the pair of lenses includes a standard optometric examination, determining which eye has the better tolerance to myopic defocusing and selecting a lens for each eye from a respective series of the range according to the results of the examination.

### BACKGROUND OF THE INVENTION

1. Field of the Invention

The invention relates to ophthalmic lenses for correcting presbyopia.

2. Description of the Prior Art

Presbyopia is a failure of accommodation of the natural lens that occurs with advancing age and requires a correction for near vision that is generally referred to as an “addition”. This is known in the art.

It is also known in the art that it is possible to correct presbyopia by fitting each eye with an ophthalmic lens, i.e. a contact lens or an intraocular implant, and that there are various solutions to enable the wearer to see clearly an object at any distance, whether that object is near, far away or at an intermediate distance.

In particular, there are lenses based on the optical principle of simultaneous vision, whereby the correcting power varies as a function of the distance from the optical axis so that a plurality of images are formed simultaneously on the retina. The wanted image is selected by cortical sorting.

French patent 2 462 854 and U.S. Pat. Nos. 5,530,491 and 5,699,141 describe simultaneous vision ophthalmic lenses in detail. The lenses described are also progressive, i.e. all the power variations are gentle, rather than sudden. The power is distributed as a function of the distance from the optical axis in accordance with a progressive profile inscribed between a lower envelope curve and an upper envelope curve, each of which curves has a polynomial expression.

This type of lens preferably has the maximum power at the center, so that near vision, which requires an addition of power because of presbyopia, uses the center of the correcting portion of the lens, while distant vision uses the periphery of the correcting portion, which is generally beyond 2 mm from the optical axis, the power being generally constant or substantially constant in the peripheral part.

Lenses with central near vision exploit the phenomenon of proximity myosis whereby, when a wearer observes a near object, for example when reading, the diameter of the pupil is reduced compared to the diameter when the wearer is observing a distant object: thus when the wearer is observing a near object they are using essentially the central area of the correcting portion of the lens, which corrects near vision, and when the wearer is observing a distant object they are using the whole of the correcting portion, and in particular its peripheral area, which produces the wanted image for distant vision.

In practice, progressive simultaneous vision ophthalmic lenses are therefore preferably sold with a central near vision area, with the facility to choose the following characteristics:

the inside radius of curvature and/or the total diameter of the lens, according to the geometry of the cornea of the eye to which the lens is to be fitted,

the power needed to correct distant vision of the eye to be fitted with the lens, i.e. the power needed to correct myopia or hypermetropia of the eye, and

the amplitude and the distribution in terms of distance from the optical axis of the power difference of the lens relative to the power needed to correct distant vision, i.e. the standard progressive profile of the lens, chosen according to the addition required by the wearer for near vision.

Thus three to four different standard progressive profiles are generally offered, for example corresponding to additions of 1.25 diopters, 2.00 diopters and 2.75 diopters, with the power varying with the distance from the optical axis in a manner that suits the huge majority of wearers.

There are nevertheless wearers whose pupillary characteristics are very different from the average.

To achieve satisfactory correction for such wearers, it has already been proposed, and in particular in U.S. Pat. No. 5,530,491, already referred to, that in particular the central area dedicated to near vision be adjusted to suit the wearer.

Three different types of power distribution can be provided to enable this, for example.

In this case, if three variation amplitudes corresponding to particular addition values are also offered, as in the above example, in total nine different standard progressive profiles must be offered.

As a result, given the necessity to vary also the power needed for the correction of myopia or hypermetropia of the eye, and the facility to choose the inside radius of curvature and/or the total diameter of the lens, the range to be offered to achieve satisfactory correction for almost all wearers comprises an extremely large number of different lenses and therefore requires a vast and costly stock to be held.

The invention aims, in contrast, to provide virtually all presbyopic persons with optimum correction using a range comprising a limited number of different lenses.

### SUMMARY OF THE INVENTION

To this end, a first aspect of the invention proposes a pair of progressive simultaneous vision ophthalmic lenses for correcting the vision of a presbyopic wearer, comprising a first lens for correcting the vision of a first eye of the wearer and a second lens for correcting the vision of their second eye, each of the first and second lenses having a correcting portion whose power, excluding any astigmatism correction, varies as a function of the distance from the optical axis in accordance with a respective progressive profile inscribed in an area between a lower envelope curve and an upper envelope curve, each envelope curve corresponding to a respective predetermined polynomial expression, in which lens pair:

for the first lens the progressive profile in accordance with which its power varies, excluding any astigmatism correction, as a function of the distance from the optical axis is such that the power is greater at a distance of 0.4 mm than at a distance of 2 mm from the optical axis and such that the power at distances from 2 mm to 2.4 mm from the optical axis does not vary by more than 0.5 diopter, and

for the second lens the progressive profile in accordance with which its power varies, excluding any astigmatism correction, as a function of the distance from the optical axis is such that the power is less at a distance of 0.4 mm than at a distance of 2 mm from the optical axis and such that the power at distances from 2 mm to 2.4 mm from the optical axis does not vary by more than 0.5 diopter.

Accordingly, in contrast to all pairs of progressive simultaneous vision ophthalmic lenses known in the art, in which both lenses have the power vary in the same sense from the center toward the periphery, preferably decreasing, the powers of the two lenses of a pair in accordance with the invention vary in opposite senses, decreasing from the center toward the periphery of the correcting portion of the first lens and increasing for the second lens.

Given that the power is constant or virtually constant at the periphery of the correcting portion of each lens, the first lens does not disturb distant vision much and the second lens does not disturb near vision much, with the result that the performance of the first and second lenses are always satisfactory for distant vision and near vision, respectively, regardless of the wearer.

The pair of lenses according to the invention therefore guarantees for almost all presbyopic wearers satisfactory visual acuity for near vision and distant vision, and because the lenses are both of the progressive simultaneous vision type, the pair of lenses also achieves very good correction of vision at intermediate distances, with the result that a wearer of the pair of lenses in accordance with the invention has good vision at all distances.

In the case of a pair of lenses using the single vision method of compensating presbyopia, which entails fitting one eye with a lens correcting only distant vision and the other eye with a lens correcting only near vision, it should be noted that the pair of lenses in accordance with the invention has the advantage of correcting vision at intermediate distances and also of avoiding, or at least significantly reducing, the binocular discomfort effects of single vision lenses caused by the fact that the difference in power between the two eyes sometimes has an inhibiting effect on essential binocular functions such as stereoscopic vision.

It appears that binocular discomfort is eliminated or reduced because the progressive simultaneous vision optics reduce perceived focusing differences between the two eyes by increasing the depth of field of the eye.

Compared to progressive simultaneous vision ophthalmic lenses known in the art, in a pair of lenses according to the invention the amplitude of the power difference between the center and the periphery of the correcting portion and the distribution of that power difference as a function of the distance from the optical axis, i.e. the standard progressive profile of the lens, do not have to be chosen to suit the addition required by the wearer for near vision, but to the contrary the first lens can have a single standard progressive profile regardless of the wearer, and likewise the second lens.

In a pair of lenses according to the invention, the addition needed for the wearer is taken into account not by choosing a standard progressive profile but instead by choosing the power of the second lens at the periphery of its correcting portion, which power is in practice made equal to the sum of the addition needed for the wearer and the power needed to correct any myopia or hypermetropia of the eye that is to receive the second lens.

The invention therefore offers the facility to correct the vision of any presbyopic wearer, regardless of the addition required, with only two different standard progressive profiles, respectively one profile for the first eye and one profile for the second eye of the wearer.

The corresponding range of lenses can therefore be particularly small, since it is sufficient for it to include a series of lenses of a first type whose respective progressive profiles vary by a predetermined power increment, the profiles being such that the power is higher at the center than at the periphery of the correcting portion, and a series of lenses of a second type whose respective progressive profiles also vary with a predetermined power increment, for example the same increment as for the series of lenses of the first type, the profiles being such that the power is lower at the center than at the periphery of the correcting portion.

In accordance with features which are preferred because of the quality of the results obtained, for each of the first and second lenses:

excluding any astigmatism correction, the absolute power difference for distances from the optical axis from 0.4 mm to 2.4 mm is at least 1 diopter,

excluding any astigmatism correction, the power varies by at most 5 diopters per millimeter at a distance of 1 mm from the optical axis, and/or

excluding any astigmatism correction, the power varies by at most 1 diopter per millimeter at a distance of 2 mm from the optical axis.

In accordance with other features which are also preferred because of the quality of the results obtained:

for the first lens, excluding any astigmatism correction, the progressive profile in accordance with which the power varies as a function of the distance from the optical axis is inscribed between a lower envelope curve and an upper envelope curve respectively represented by the following equations:

P1₁(h)=P_{VL1}+A (h)−0.18

P1ᵤ(h)=P1₁ (h)+0.36

for the second lens, excluding any astigmatism correction, the progressive profile in accordance with which the power varies as a function of the distance from the optical axis is inscribed between a lower envelope curve and an upper envelope curve respectively represented by the following equations:

P2₁(h)=P_{VL2}+P_{ADD}−B(h)−0.18

P2ᵤ(h)=P2₁(h)+0.36

and, in the equations:

P_{VL1} and P_{VL2} are the powers expressed in diopters (D), excluding any astigmatism correction, that may be needed to correct near vision for the first eye and for the second eye, respectively,

P_{ADD} is the addition, expressed in diopters (D), required by the wearer for near vision,

h is the distance from the optical axis expressed in millimeters (mm), and

A (h) is equal to

$${\sum }_{i = 0}^{i = 9} {α}_{2 i} {h}^{2 i}$$

and B (h) is equal to

$${\sum }_{i = 0}^{i = 9} {β}_{2 i} {h}^{2 i} ,$$

for values of h from 0.4 mm to 2.4 mm, the series of coefficients α₂ᵢ and β₂ᵢ, for i from 1 to 9, defined by a respective one of the following nine of coefficients SA, SB, SC, MA, MB, MC, LA, LB, LC:

| i | SA | SB | SC |
|---|---|---|---|
| 0 | 1.398800E+00 | 3.093330E+00 | 4.605640E+00 |
| 1 | −2.160020E+00 | −4.751140E+00 | 5.235240E+00 |
| 2 | 1.337720E+00 | 2.913630E+00 | 2.458240E+00 |
| 3 | −4.327890E−01 | −9.378340E−01 | 6.301520E−01 |
| 4 | 8.154230E−02 | 1.764900E−01 | 9.787570E−02 |
| 5 | −9.410290E−03 | −2.038990E−02 | 9.616130E−03 |
| 6 | 6.736380E−04 | 1.462890E−03 | 6.012020E−04 |
| 7 | −2.914960E−05 | −6.347570E−05 | 2.318560E−05 |
| 8 | 6.978470E−07 | 1.520000E−06 | 5.030000E−07 |
| 9 | −7.091930E−09 | −1.550000E−08 | 4.690000E−09 |
| i | MA | MB | MC |
| 0 | 1.799020E+00 | 3.048790E+00 | 4.144890E+00 |
| 1 | −1.823880E+00 | −3.424400E+00 | −4.233760E+00 |
| 2 | 8.133470E−01 | 1.714210E+00 | 1.949870E+00 |
| 3 | −2.057150E−01 | −4.850380E−01 | −5.212190E−01 |
| 4 | 3.222470E−02 | 8.400400E−02 | 8.739800E−02 |
| 5 | −3.231690E−03 | −9.184070E−03 | −9.410210E−03 |
| 6 | 2.075120E−04 | 6.343800E−04 | 6.468110E−04 |
| 7 | −8.241900E−06 | −2.679260E−05 | −2.734250E−05 |
| 8 | 1.842050E−07 | 6.310000E−07 | 6.460000E−07 |
| 9 | −1.770040E−09 | −6.330000E−09 | −6.520000E−09 |
| i | LA | LB | LC |
| 0 | 1.258120E+00 | 2.3409009E+00 | 2.660000E+00 |
| 1 | 2.766510E−01 | −1.6016233E+00 | −3.029760E−00 |
| 2 | −5.863900E−01 | 8.5580090E−01 | 1.837526E−60 |
| 3 | 2.158210E−01 | −4.0855924E−01 | −6.361990E−61 |
| 4 | −3.890640E−02 | 1.2233248E−01 | 1.293966E−01 |
| 5 | 4.063430E−03 | −2.1406740E−02 | −1.595350E−62 |
| 6 | −2.578890E−04 | 2.2148862E−03 | 1.265296E−03 |
| 7 | 9.821560E−06 | −1.3380186E−04 | −5.450006E−05 |
| 8 | −2.065710E−07 | 4.3658573E−06 | 1.350000E−06 |
| 9 | 1.845210E−09 | −5.9468409E−08 | −1.410000E−08 |

in which lists E and the number after it represent a of 10.

In a first preferred embodiment the functions A(h) and B(h) are identical and the coefficients α₂ᵢ and β₂ᵢ are from the same list.

In a second preferred embodiment the functions A(h) and B(h) are different and the coefficients α₂ᵢ and β₂ᵢ are from two different lists.

According to other preferred features the correcting portion of at least one of the first and second lenses also corrects astigmatism.

A second aspect of the invention provides a range of progressive simultaneous vision ophthalmic lenses including a series of lenses of a first type and a series of lenses of a second type for making up a pair of opthalmic lenses for correcting the vision of a presbyopic wearer with a first lens for correcting the vision of a first eye of the wearer taken from the series of lenses of the first type and a second lens for correcting the vision of the second eye of the wearer taken from the series of lenses of a second type, in which range of lenses:

each lens from the series of lenses of the first type and from the series of lenses of the second type has a correcting portion whose power, excluding any astigmatism correction, varies as a function of the distance from the optical axis in accordance with a respective progressive profile inscribed in an area between a lower envelope curve and an upper envelope curve, each envelope curve having a respective predetermined polynomial expression, the respective profiles of the lenses of the series of lenses of the first varying with a predetermined power increment, and likewise for the series of lenses of the second type,

for each lens from the series of lenses of the first type the progressive profile in accordance with which its power varies, excluding any astigmatism correction, as a function of the distance from the optical axis is such that the power is greater at a distance of 0.4 mm than at a distance of 2 mm from the optical axis and such that the power at distances from 2 mm to 2.4 mm from the optical axis does not vary by more than 0.5 diopter, and

for each lens from the series of lenses of the second type the progressive profile in accordance with which its power varies, excluding any astigmatism correction, as a function of the distance from the optical axis is such that the power is less at a distance of 0.4 mm than at a distance of 2 mm from the optical axis and such that the power at distances from 2 mm to 2.4 mm from the optical axis does not vary by more than 0.5 diopter.

According to specific preferred features of the range according to the invention:

for each lens from the series of lenses of the first type, excluding any astigmatism correction, the progressive profile in accordance with which the power varies as a function of the distance from the optical axis is inscribed between a lower envelope curve and an upper envelope curve respectively represented by the following equations:

P1₁(h)=Pₙ+A(h)−0.18

P1ᵤ(h)=P1₁(h)+0.36

for each lens from the series of lenses of the second type, excluding any astigmatism correction, the progressive profile in accordance with which the power varies as a function of the distance from the optical axis is inscribed between a lower envelope curve and an upper envelope curve respectively represented by the following equations:

P2₁(h)=Pₘ−B(h)−0.18

P2ᵤ(h)=P2₁(h)+0.36

and, in the equations:

Pₙ is a power expressed in diopters (D) which changes from one lens to another of the series of lenses of the first type with the predetermined increment,

Pₘ is a power expressed in diopters (D) which changes from one lens to another of the series of lenses of the second type with the predetermined increment,

h is the distance from the optical axis expressed in millimeters (mm), and

A (h) is equal to

$${\sum }_{i = 0}^{i = 9} {α}_{2 i} {h}^{2 i}$$

and B (h) is equal to

$${\sum }_{i = 0}^{i = 9} {β}_{2 i} {h}^{2 i} ,$$

for values of h from 0.4 mm to 2.4 mm, the series of coefficients α₂ᵢ and β₂ᵢ, for i from 1 to 9, being defined by a respective one of the above nine lists of coefficients SA, SB, SC, MA, MB, MC, LA, LB, LC.

Finally, a third aspect of the invention provides a method of obtaining a pair of progressive simultaneous vision ophthalmic lenses for correcting the vision of a presbyopic wearer, including the following steps:

a) a step of determining the addition needed for the wearer and the power needed for each eye of the wearer to correct any myopia or hypermetropia,

b) a step of determining which eye of the wearer, referred to as the second eye, has the better tolerance for myopic defocusing, i.e. the blurring introduced by a lens having a positive power,

c) a step of selecting, from the above range of progressive simultaneous vision ophthalmic lenses, a lens from the series of lenses of the first type whose power Pₙ is equal to the power needed to correct any myopia or hypermetropia of the first eye of the wearer, and

d) a step of selecting from the range of lenses a lens from the series of lenses of the second type whose power Pₘ is equal to the sum of the power needed to correct any myopia or hypermetropia of the second eye of the wearer and the addition of the wearer.

Note that step b) offers the advantage of minimizing any discomfort that the wearer might feel because the second lens has at the periphery a power corresponding to the power needed to correct any myopia or hypermetropia plus the addition required to correct their presbyopia.

In accordance with other preferred features, the method according to the invention further includes the following optimization steps intended to achieve the best possible vision for the wearer:

a step of determining the lens from the series of lenses of the first type whose power Pₙ for distant vision is the highest possible tolerated by the wearer,

a step of determining the lens from the series of lenses of the second type whose power Pₘ for near vision is the lowest tolerated by the wearer, and

repeating the preceding two steps alternately, if required, until the best compromise is arrived at.

The optimization steps are preferably conducted for binocular vision.

The explanation of the invention will now continue with a description of preferred embodiments of the invention given hereinafter by way of illustrative and non-limiting example and with reference to the accompanying drawings.

### BRIEF DESCRIPTION OF THE DRAWINGS

FIG. 1 is a diagram showing a range of ophthalmic lenses according to the invention.

FIG. 2 is a front view of a pair of ophthalmic lenses according to the invention.

FIG. 3 is a diagrammatic view of part of one of the lenses in axial section.

FIGS. 4 and 5 are diagrams representing the power of respective lenses of the pair as a function of the distance from the optical axis.

FIGS. 6 and 7 are diagrams similar to FIGS. 4 and 5 for a different embodiment of the pair of lenses, in which the lenses have different standard profiles.

FIGS. 8 to 14 are diagrams showing different examples of standard profiles for implementing the invention, each diagram showing in particular the envelope curves between which the standard profile must be inscribed.

### DETAILED DESCRIPTION OF THE PREFERRED EMBODIMENTS

The range 1 of ophthalmic lenses shown in FIG. 1 includes a series 2 of lenses 2A, 2B, . . . , 2Z of a first type and a series 3 of lenses 3A, 3B, . . . , 3Z of a second type.

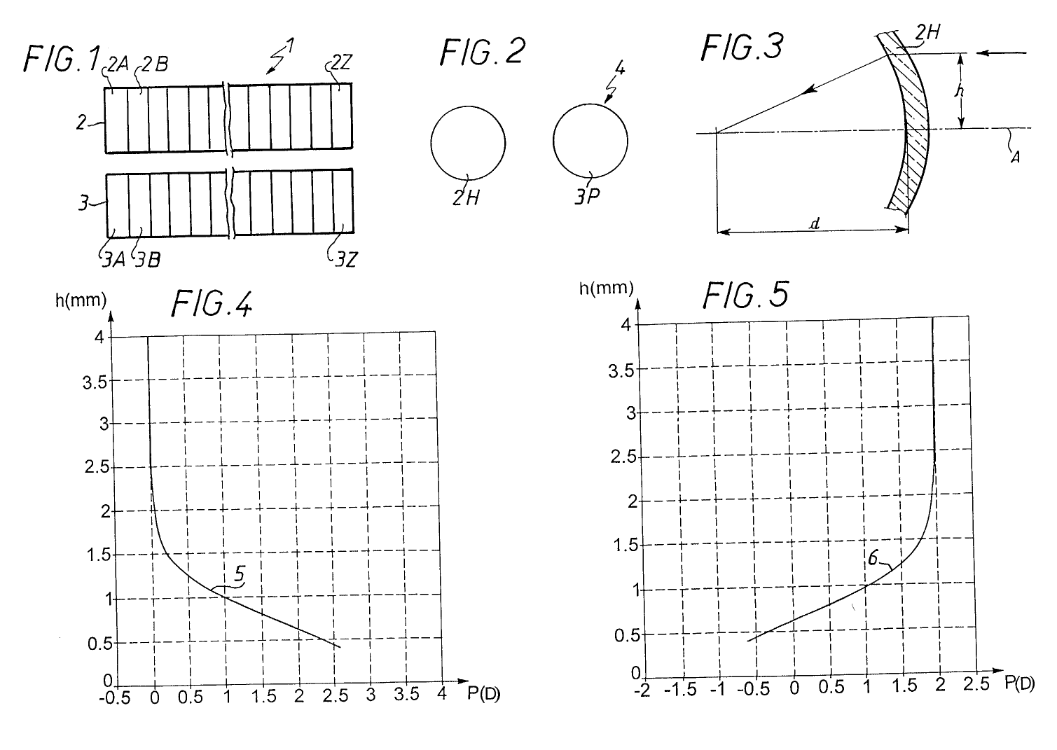

The range 1 enables a pair of ophthalmic lenses to be prescribed to correct the vision of a presbyopic wearer, such as the pair 4 shown in FIG. 2, which includes a lens 2H from the series 2 of lenses of the first type, for correcting the vision of a first eye of the wearer, here the left eye, and a lens 3P from the series 3 of lenses of the second type, for correcting the vision of the other eye of the wearer, here the right eye.

Each of the lenses from the range 1, and therefore the lens 2H shown in FIG. 3, is circular and has a central optical axis A and a correcting portion between the axis A and points at a distance of 4 mm from that axis.

Broadly speaking, because the lens 2H shown is a convergent lens, any incident light ray parallel to the axis A and at a distance h from that axis intersects the axis A at a point d from the lens 2H after passing through it.

The power P expressed in diopters (D) is broadly defined as the reciprocal of the distance d expressed in meters.

To be more precise, in the present context, the power P is defined as the sagittal power

$$P ( h ) = \frac{1}{h} \frac{d ( δ ( h )}{dh} ,$$

where δ(h) is the optical path difference introduced by the lens for a light ray parallel to the optical axis and at a distance h therefrom and is related to the phase-shift caused by the lens by the equation

$$ϕ ( h ) = \frac{2 π δ ( h )}{λ} ,$$

where δ(h) is the phase-shift at the distance h and λ is the wavelength of the light ray, negative values of δ and φ corresponding to a time-delay applied to the optical wave and positive values to an advance. P is expressed in diopters (D), h in millimeters (mm), δ and λ in micrometers (μm) and φ in radians (rad), for example.

In practice, φ(h) can be determined by interferometry or by some other method of measuring optical phase-shift.

Each lens from the range 1 is of the progressive simultaneous vision type, and as a result the power of each lens varies gently between the center and the periphery of the optical area.

To be more precise, each lens from the range 1 has a standard progressive profile between the circle of radius h=0.4 mm and the circle of radius h=2.4 mm.

Note that if the illumination is good and the lens is perfectly centered, light rays passing through the pupil pass through the lens in an area between the optical axis A and a circle of radius h=2.4 mm and that if the above conditions are not satisfied rays passing through the lens in the area between h=2.4 mm and the edge of the correcting area also pass through the pupil.

In the preferred examples shown and described, the power is constant between the circles h=2.4 mm and h=4 mm, but the power can instead vary in this area.

Note also that because of practical fabrication and measurement difficulties, the power values for the area between the axis A and the circle of radius h=0.4 mm, which represents a very small proportion (a few %) of the area of the pupil, are not significant, and so those power values are not referred to or shown in the description or the accompanying drawing.

FIG. 4 shows that the progressive profile 5 in accordance with which the power of the lens 2H varies as a function of h is such that the power is zero (no correction) between h=4 mm and h=2.4 mm and then increases progressively between h=2.4 mm and h=0.4 mm. The power is approximately 2.6 diopters on the circle h=0.4 mm.

The profile 6 of the lens 3P shown in FIG. 5 is such that the power is constant between the circles of radius h=4 mm and h=2.4 mm and then decreases progressively to approximately −0.6 diopter on the circle h=0.4 mm.

Note that the profile 6 corresponds to the mirror image of the profile 5, shifted by +2 diopters. In other words, the profile 6, expressed in the form of a function g(h), is deduced from the profile 5 expressed in the form of a function f(h) by the equation g(h)=2−f(h).

The lens pair 4 is intended to correct the vision of a presbyopic wearer who requires an addition of 2 diopters and does not suffer from myopia, hypermetropia or astigmatism.

It can be seen that the lens 2H applies no correction or virtually no correction to the left eye of the wearer in the peripheral area of the correcting portion, situated beyond h=2 mm, whereas the lens 3P applies a constant or virtually constant correction of +2 diopters to the right eye in the peripheral area of the correcting portion, this being the value required to correct the wearer's presbyopia, with the result that the lens 2H features central near vision correction disturbing distant vision only slightly and the lens 3P features central distant vision correction disturbing near vision only slightly.

The wearer of the lens pair 4 therefore has satisfactory visual acuity at all distances, i.e. for near vision, intermediate vision and distant vision, and binocular vision is also satisfactory.

To obtain the lens pair 4 suited to the wearer, i.e. to determine that it is the lenses 2H and 3P from the range 1 that suit the wearer, a standard optometric examination is first carried out to determine, in particular by refraction, the power needed to correct any myopia or hypermetropia of each eye, and the addition needed for the wearer concerned. In this example the examination indicates that no correction of myopia or hypermetropia is required and that the addition needed is 2 diopters.

The examination also determines which of the two eyes has the better tolerance to myopic defocusing, i.e. to the blurring introduced by the lens having a positive power. The examination shows that it is the right eye which has the better tolerance.

The lens 3P from the series 3, each lens in which has a progressive profile deduced from a standard profile provided for the eye having the better tolerance to myopic defocusing, which corresponds to a nominal power of 2 diopters, this being the sum of the power needed to correct myopia or hypermetropia, which is zero in this example, and the addition required by the wearer, i.e. 2 diopters.

The lens 2H is selected from the series 2, the lenses in which have a profile corresponding to a standard profile provided for the eye that does not have the better tolerance of myopic defocusing, which lens has a nominal power of 0 diopter, the left eye requiring no correction for myopia or hypermetropia.

When the lenses have been chosen and placed on the eyes of the wearer, optimization steps are carried out using trial lenses and binocular vision to find, for the left eye (which has the lower tolerance of myopic defocusing), the highest possible power in near vision tolerated by the wearer, and for the right eye (which has the better tolerance of myopic defocusing), the lowest possible power in near vision tolerated by the wearer. Alternating adjustments finally show that the best compromise is that retaining the nominal powers of 0 diopter for the left eye and +2 diopters for the right eye.

Two further examples will now be given, to explain how to determine, from the range 1, the lenses needed to correct presbyopia, and possibly myopia or hypermetropia, of virtually all presbyopic wearers, the series 2 of lenses (for the eye with the lower tolerance for myopic defocusing) including lenses whose nominal power runs from −20.00 to +20.00 diopters in steps of 0.25 diopter, and the series 3 of lenses (for the eye with the better tolerance for myopic defocusing) including lenses whose nominal power runs from −19.00 to +23.00 diopters in steps of 0.25 diopter.

The first of the two examples relates to a relatively elderly wearer who is severely myopic, the optometric examination showing that the left eye requires a correction of −9.00 diopters, the right eye requires a correction of −11.00 diopters and the addition needed for the wearer concerned is 3 diopters.

The myopic defocusing tolerance examination shows that the right eye has the better tolerance.

The lens of nominal power −9.00 diopters is therefore chosen for the left eye from the series 2 and the lens having the nominal power of −8.00 diopters (−11.00++3.00) is chosen for the right eye from the series 3, the corresponding lenses being placed on the eyes of the patient before carrying out the optimization steps referred to above using trial lenses.

The second additional example relates to a relatively young wearer suffering from hypermetropia and for whom the optometric examination determines that the power needed to correct the hypermetropia is +3 diopters for the right eye and +5 diopters for the left eye, this wearer requiring an addition of +1.25 diopters.

The myopic defocusing tolerance examination shows that the left eye has the greater tolerance and a lens having a nominal power of 6.25 diopters (+5.00++1.25) is chosen from the series 3 of lenses for the left eye and a lens having a nominal power of +3.00 diopters is chosen from the series 2 of lenses for the right eye.

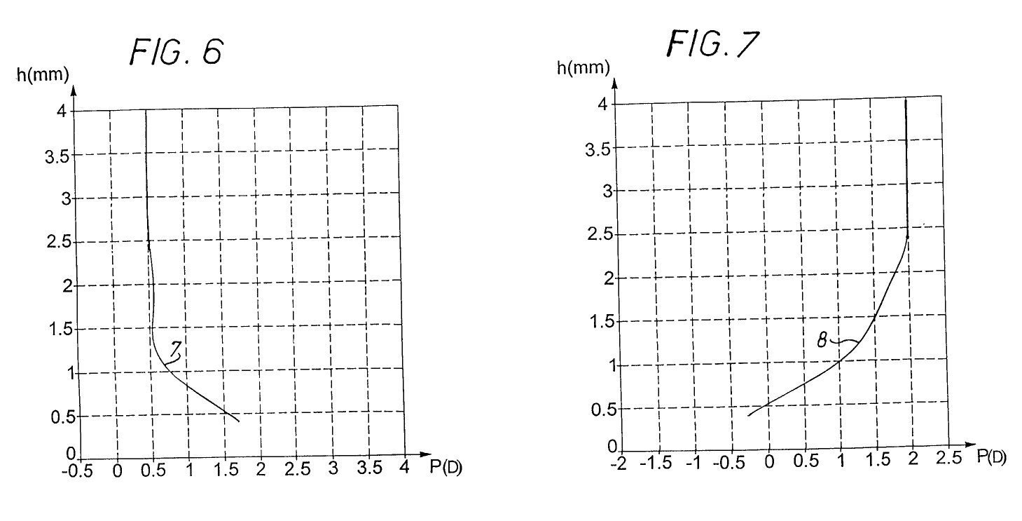

A variant of the range 1 in which the standard profile used for the lenses of one series is not the mirror image of the standard profile used for the other series will now be described with reference to FIGS. 6 and 7.

The two lenses respectively having the progressive profile 7 shown in FIG. 6 and the progressive profile 8 shown in FIG. 7 are respectively intended for the left eye and the right eye. The examination shows that the right eye has the better tolerance for myopic defocusing, the left eye suffers from slight hypermetropia and requires a correction of 0.5 diopter and the right eye requires no correction, this wearer requiring an addition of 2 diopters.

It can be seen that the manner in which the profile 7 varies between the circles of radius h=2.4 mm and h=0.4 mm is very different from how the corresponding portion of the profile 8 varies, and that in particular the variation is approximately 1.2 diopters for the profile 7 and approximately 2.3 diopters for the profile 8, and that the power variation is relatively slight for the profile 7 between the circles h=2.4 mm and h=1.4 mm while for the profile 8 the power varies by approximately 0.6 diopter over the same area.

Generally speaking, the trials conducted have shown that the profiles described hereinafter yield entirely satisfactory results:

for each lens from one series of the range, for example the series 2 of the range 1, the progressive profile by which the power varies as a function of h is inscribed between a lower envelope curve and an upper envelope curve respectively represented by the equations:

P1₁(h)=Pₙ+A(h)−0.18

P1ᵤ(h)=P1₁(h)+0.36

for each lens from the other series of the range, for example the series 3 of the range 1, the progressive profile by which the power varies as a function of h is inscribed between a lower envelope curve and an upper envelope curve respectively represented by the equations:

P2₁(h)=Pₘ−B(h)−0.18

P2ᵤ(h)=P2₁(h)+0.36

In the above equations:

Pₙ is a power, expressed in diopters (D), which changes from one lens to the other of the first series of lenses with a predetermined increment,

Pₘ is a power, expressed in diopters (D), which changes from one lens to the other of the second series lenses with a predetermined increment, and

A (h) is equal to

$${\sum }_{i = 0}^{i = 9} {α}_{2 i} {h}^{2 i}$$

and B (h) is equal to

$${\sum }_{i = 0}^{i = 9} {β}_{2 i} {h}^{2 i} ,$$

for values of h from 0.4 mm to 2.4 mm, the series of coefficients α₂ᵢ and the series of coefficients β₂ᵢ, for i from 1 to 9, each being defined by a respective one of the following nine lists SA, SB, SC, MA, MB, MC, LA, LB, LC of coefficients:

| i | SA | SB | SC |
|---|---|---|---|
| 0 | 1.398800E+00 | 3.093330E+00 | 4.605640E+00 |
| 1 | −2.160020E+00 | −4.751140E+00 | −5.235240E+00 |
| 2 | 1.337720E+00 | 2.913640E+00 | 2.458240E+00 |
| 3 | −4.327890E−01 | −9.378340E−01 | −6.301520E−01 |
| 4 | 8.154230E−02 | 1.764900E−01 | 9.787570E−02 |
| 5 | −9.410290E−03 | −2.038990E−02 | −9.616130E−03 |
| 6 | 6.736380E−04 | 1.462890E−03 | 6.012020E−04 |
| 7 | −2.914960E−05 | −6.347570E−05 | −2.318560E−05 |
| 8 | 6.978470E−07 | 1.520000E−06 | 5.030000E−07 |
| 9 | −7.091930E−09 | −1.550000E−08 | −4.690000E−09 |
| i | MA | MB | MC |
| 0 | 1.799020E+00 | 3.048790E+00 | 4.144890E+00 |
| 1 | −1.823880E+00 | −3.424400E+00 | −4.233760E+00 |
| 2 | 8.133470E−01 | 1.714210E+00 | 1.949870E−00 |
| 3 | −2.057150E−01 | −4.850380E−01 | −5.212190E−01 |
| 4 | 3.222470E−02 | 8.400400E−02 | 8.739800E−02 |
| 5 | −3.231690E−03 | −9.184070E−03 | −9.410210E−03 |
| 6 | 2.075120E−04 | 6.343800E−04 | 6.468110E−04 |
| 7 | −8.241900E−06 | −2.679260E−05 | −2.734250E−05 |
| 8 | 1.842050E−07 | 6.310000E−07 | 6.460000E−07 |
| 9 | −1.770040E−09 | −6.330000E−09 | −6.520000E−09 |
| i | LA | LB | LC |
| 0 | 1.258120E+00 | 2.3409009E+00 | 2.660000E+00 |
| 1 | 2.766510E−01 | −1.6016233E+00 | −3.029760E+00 |
| 2 | −5.863900E−01 | 8.5580090E−01 | 1.837520E+00 |
| 3 | 2.158210E−01 | −4.0855924E−01 | −6.361990E−01 |
| 4 | −3.890640E−02 | 1.2233248E−01 | 1.293960E−01 |
| 5 | 4.063430E−03 | −2.1406740E−02 | −1.595350E−02 |
| 6 | −2.578890E−04 | 2.2148862E−03 | 1.205290E−03 |
| 7 | 9.821560E−06 | −1.3380186E−04 | −5.450000E−05 |
| 8 | −2.065710E−07 | 4.3658573E−06 | 1.350000E−06 |
| 9 | 1.845210E−09 | −5.9468409E−08 | −1.410000E−08 |

In the above lists, E and the number after it represent a power of 10.

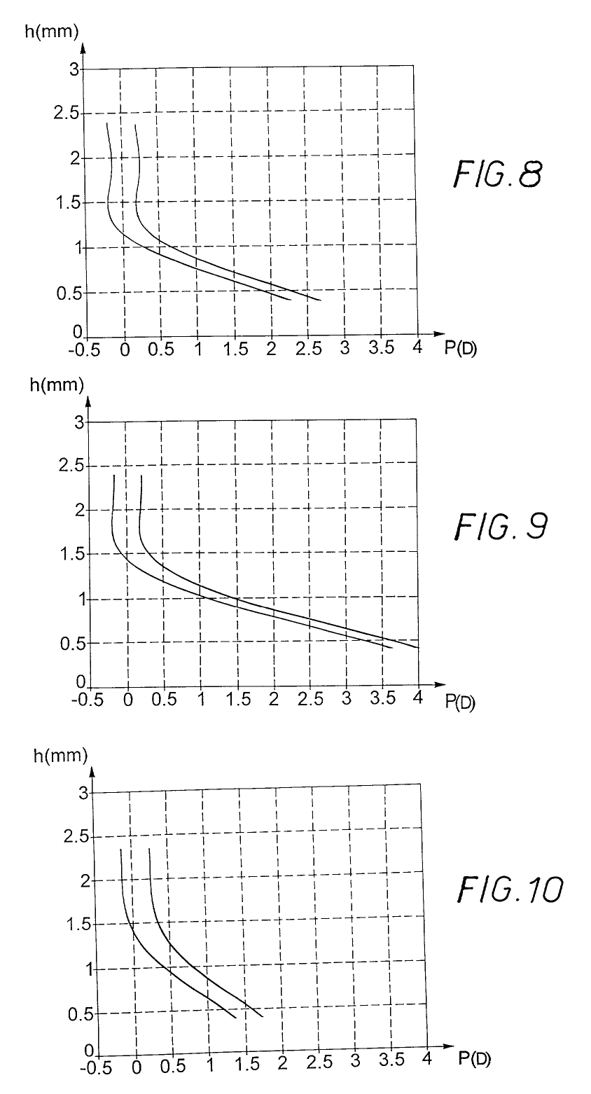

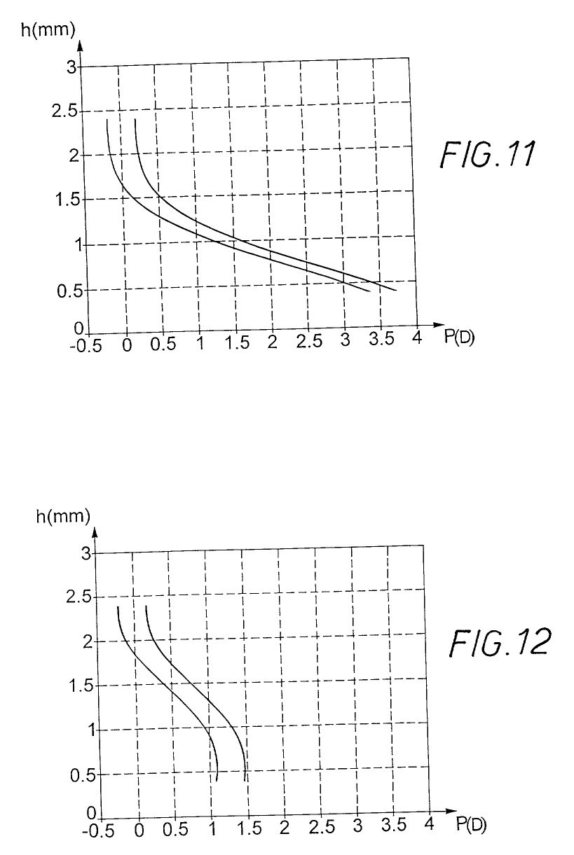

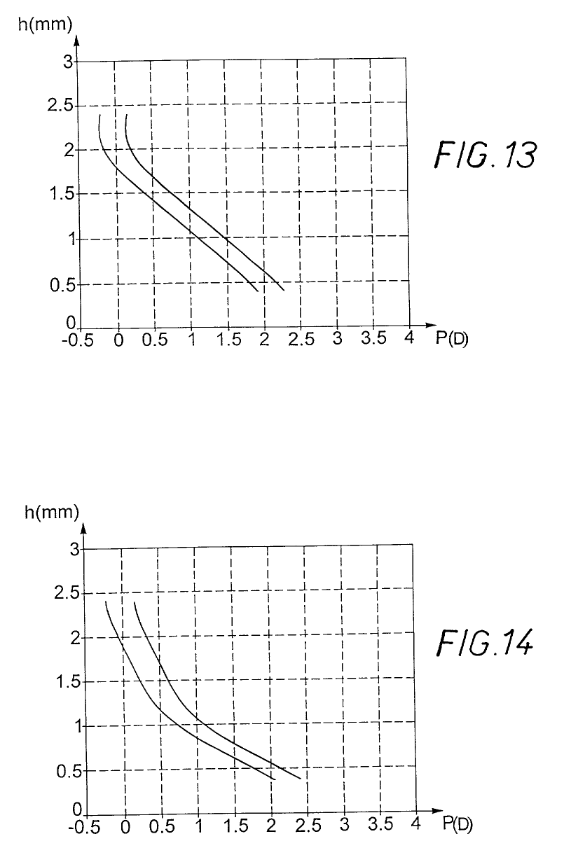

FIGS. 8 to 14 show respectively the lower envelope curve and the upper envelope curve of the standard profiles, i.e. P1₁(h) and P1ᵤ(h), Pₙ is equal to 0 and h varies from 0.4 mm to 2.4 mm.

To be more precise, FIGS. 8 to 14 respectively show the profiles corresponding to the tables of coefficients SB, SC, MA, MC, LA, LB and LC.

Note that the profiles 5 and 6 shown in FIGS. 4 and 5 correspond to the standard profiles given by the table of coefficients MB and the profiles 7 and 8 shown in FIGS. 6 and 7 respectively correspond to the standard profiles given by the tables of coefficients SA and LC.

Of course, the first series of lenses is intended for the eye having the lower tolerance for myopic defocusing and the second series of lenses is intended for the other eye. Pₙ is the power needed to correct any myopia or hypermetropia of the eye having the lower tolerance for myopic defocusing and Pₘ is the sum of any power needed to correct any myopia or hypermetropia of the eye having the better tolerance to myopic defocusing and the addition required by the wearer.

Note that at present the best combinations would seem to be as follows:

| | 1 | 2 | 3 | 4 |
|---|---|---|---|---|
| List for eye with lower | MA | LC | LB | MA |
| tolerance for myopic defocusing | | | | |
| List for the other eye | LC | LC | LB | MA |

and that of these four combinations, that offering the best performance would appear to be combination 3.

Note that for each standard profile the power does not vary by more than 0.5 diopter beyond h=2 mm, the greatest increase being shown in FIG. 14 (table of coefficients LC), that the power slope at h=1 mm remains below 5 diopters per millimeter, and that the slope remains less than 1 diopter per millimeter at h=2 mm.

In an embodiment of the invention that is not shown the lenses described above correct not only presbyopia and possibly myopia or hypermetropia but also astigmatism, thanks to a correction having toric characteristics.

More generally, many embodiments are available to suit differing circumstances and in this connection it should be borne in mind that the invention is not limited to the examples shown and described.

There is claimed:

1. A pair of progressive simultaneous vision ophthalmic lenses for correcting the vision of a presbyopic wearer, comprising a first lens for correcting the vision of a first eye of said wearer and a second lens for correcting the vision of their second eye, each of said first and second lenses having a correcting portion whose power, excluding any astigmatism correction, varies as a function of the distance from the optical axis in accordance with a respective progressive profile inscribed in an area between a lower envelope curve and an upper envelope curve, each envelope curve corresponding to a respective predetermined polynomial expression, in which lens pair:
for said first lens the progressive profile in accordance with which its power, excluding any astigmatism correction, varies as a function of said distance from said optical axis is such that said power is greater at a distance of 0.4 mm than at a distance of 2 mm from said optical axis and such that said power at distances from 2 mm to 2.4 mm from said optical axis does not vary by more than 0.5 diopter, and
for said second lens the progressive profile in accordance with which its power, excluding any astigmatism correction, varies as a function of said distance from said optical axis is such that said power is less at a distance of 0.4 mm than at a distance of 2 mm from said optical axis and such that said power at distances from 2 mm to 2.4 mm from said optical axis does not vary by more than 0.5 diopter.

2. The pair of lenses claimed in claim 1 wherein, excluding any astigmatism correction, the absolute power difference for each of said first and second lenses for distances from said optical axis from 0.4 mm to 2.4 mm is at least 1 diopter.

3. The pair of lenses claimed in claim 1 wherein, excluding any astigmatism correction, the power for each of said first and second lenses varies by at most 5 diopters per millimeter at a distance of 1 mm from said optical axis.

4. The pair of lenses claimed in claim 1 wherein, excluding any astigmatism correction, the power for each of said first and second lenses varies by at most 1 diopter per millimeter at a distance of 2 mm from said optical axis.

5. The pair of lenses claimed in claim 1 wherein:
for said first lens, excluding any astigmatism correction, said progressive profile in accordance with which said power varies as a function of said distance from said optical axis is inscribed between a lower envelope curve and an upper envelope curve respectively represented by the following equations:
P1₁(h)=P_{VL1}+A(h)−0.18
P1ᵤ(h)=P1₁(h)+0.36
for said second lens, excluding any astigmatism correction, said progressive profile in accordance with which said power varies as a function of said distance from said optical axis is inscribed between a lower envelope curve and an upper envelope curve respectively represented by the following equations:
P2₁(h)=P_{VL2}+P_{ADD}−B(h)−0.18
P2ᵤ(h)=P2₁(h)+0.36
and, in said equations:
P_{VL1} and P_{VL2} are the powers expressed in diopters, excluding any astigmatism correction, that may be needed to correct near vision for said first eye and for said second eye, respectively,
P_{ADD} is the addition, expressed in diopters, required by the wearer for near vision,
h is the distance from said optical axis expressed in millimeters, and
A (h) is equal to
$${\sum }_{i = 0}^{i = 9} {α}_{2 i} {h}^{2 i}$$
and B (h) is equal to
$${\sum }_{i = 0}^{i = 9} {β}_{2 i} {h}^{2 i} ,$$
for values of h from 0.4 mm to 2.4 mm, the of coefficients α₂ᵢ and β₂ᵢ, for i from 1 to 9, being defined by a respective one of the following nine lists of coefficients SA, SB, SC, MA, MB, MC, LA, LB, LC:

| i | SA | SB | SC |
|---|---|---|---|
| 0 | 1.398800E+00 | 3.093330E+00 | 4.605640E+00 |
| 1 | −2.160020E+00 | −4.751140E+00 | −5.235240E+00 |
| 2 | 1.337720E+00 | 2.913640E+00 | 2.458240E+00 |
| 3 | −4.327890E−01 | −9.378340E−01 | −6.301520E−01 |
| 4 | 8.154230E−02 | 1.764900E−01 | 9.787570E−02 |
| 5 | −9.410290E−03 | −2.038990E−02 | −9.616130E−03 |
| 6 | 6.736380E−04 | 1.462890E−03 | 6.012020E−04 |
| 7 | −2.914960E−05 | −6.347570E−05 | −2.318560E−05 |
| 8 | 6.978470E−07 | 1.520000E−06 | 5.030000E−07 |
| 9 | −7.091930E−09 | −1.550000E−08 | −4.690000E−09 |
| i | MA | MB | MC |
| 0 | 1.799020E+00 | 3.048790E+00 | 4.144890E+00 |
| 1 | −1.823880E+00 | −3.424400E+00 | −4.233760E+00 |
| 2 | 8.133470E−01 | 1.714210E+00 | 1.949870E+00 |
| 3 | −2.057150E−01 | −4.850380E−01 | −5.212190E−01 |
| 4 | 3.222470E−02 | 8.400400E−02 | 8.739800E−02 |
| 5 | −3.231690E−03 | −9.184070E−03 | −9.410210E−03 |
| 6 | 2.075120E−04 | 6.343800E−04 | 6.468110E−04 |
| 7 | −8.241900E−06 | −2.679260E−05 | −2.734250E−05 |
| 8 | 1.842050E−07 | 6.310000E−07 | 6.460000E−07 |
| 9 | −1.770040E−09 | −6.330000E−09 | −6.520000E−09 |
| i | LA | LB | LC |
| 0 | 1.258120E+00 | 2.3409009E+00 | 2.660000E+00 |
| 1 | 2.766510E−01 | −1.6016233E+00 | −3.029760E+00 |
| 2 | −5.863900E−01 | 8.5580090E−01 | 1.837520E+00 |
| 3 | 2.158210E−01 | −4.0855924E−01 | −6.361990E−01 |
| 4 | −3.890640E−02 | 1.2233248E−01 | 1.293960E−01 |
| 5 | 4.063430E−03 | −2.1406740E−02 | −1.595350E−02 |
| 6 | −2.578890E−04 | 2.2148862E−03 | 1.205290E−03 |
| 7 | 9.821560E−06 | −1.3380186E−04 | −5.450000E−05 |
| 8 | −2.065710E−07 | 4.3658573E−06 | 1.350000E−06 |
| 9 | 1.845210E−09 | −5.9468409E−08 | −1.410000E−08 |

in which lists E and the number after it represent a power of 10.

6. The pair of lenses claimed in claim 5 wherein said functions A(h) and B(h) are identical and said coefficients α₂ᵢ and β₂ᵢ are taken from the same list.

7. The pair of lenses claimed in claim 5 wherein said functions A(h) and B(h) are different and said coefficients α₂ᵢ and β₂ᵢ are taken from two different lists.

8. The pair of lenses claimed in claim 1 wherein the correcting portion of at least one of said first and second lenses also corrects astigmatism.

9. A range of progressive simultaneous vision ophthalmic lenses including a series of lenses of a first type and a series of lenses of a second type for making up a pair of ophthalmic lenses for correcting the vision of a presbyopic wearer with a first lens for correcting the vision of a first eye of said wearer taken from said series of lenses of said first type and a second lens for correcting the vision of the second eye of said wearer taken from said series of lenses of a second type, in which range of lenses:
each lens from said series of lenses of said first type and from said series of lenses of said second type has a correcting portion whose power, excluding any astigmatism correction, varies as a function of said distance from said optical axis in accordance with a respective progressive profile inscribed in an area between a lower envelope curve and an upper envelope curve, each envelope curve having a respective predetermined polynomial expression, the respective profiles of said lenses of said series of lenses of said first varying with a predetermined power increment, and likewise for said series of lenses of said second type,
for each lens from said series of lenses of said first type the progressive profile in accordance with which its power, excluding any astigmatism correction, varies as a function of said distance from said optical axis is such that said power is greater at a distance of 0.4 mm than at a distance of 2 mm from said optical axis and such that said power at distances from 2 mm to 2.4 mm from said optical axis does not vary by more than 0.5 diopter, and
for each lens from said series of lenses of said second type the progressive profile in accordance with which its power, excluding any astigmatism correction, varies as a function of said distance from said optical axis is such that said power is less at a distance of 0.4 mm than at a distance of 2 mm from said optical axis and such that said power at distances from 2 mm to 2.4 mm from said optical axis does not vary by more than 0.5 diopter.

10. The range of lenses claimed in claim 9 wherein, excluding any astigmatism correction, the absolute power difference for each lens from said series of lenses of said first type and for each lens from said series of lenses of said second type for distances from said optical axis from 0.4 mm to 2.4 mm is at least 1 diopter.

11. The range of lenses claimed in claim 9 wherein, excluding any astigmatism correction, the power for each lens from said series of lenses of said first type and for each lens from said series of lenses of said second type varies by at most 5 diopters per millimeter at a distance of 1 mm from said optical axis.

12. The range of lenses claimed in claim 9 wherein, excluding any astigmatism correction, the power for each lens from said series of lenses of said first type and for each lens from said series of lenses of said second type varies by at most 1 diopter per millimeter at a distance of 2 mm from said optical axis.

13. The range of lenses claimed in claim 9 wherein said predetermined increment is 0.25 diopter.

14. The range of lenses claimed in claim 9 including at least one lens whose correcting portion also corrects astigmatism.

15. The range of lenses claimed in claim 9 wherein:
for each lens from said series of lenses of said first type, excluding any astigmatism correction, said progressive profile in accordance with which said power varies as a function of said distance from said optical axis is inscribed between a lower envelope curve and an upper envelope curve respectively represented by the following equations:
P1₁(h)=Pₙ+A(h)−0.18
P1ᵤ(h)=P1₁(h)+0.36
for each lens from said series of lenses of said second type, excluding any astigmatism correction, said progressive profile in accordance with which said power varies as a function of said distance from said optical axis is inscribed between a lower envelope curve and an upper envelope curve respectively represented by the following equations:
P2₁(h)=Pₘ−B(h)−0.18
P2ᵤ(h)=P2₁(h)+0.36
and, in said equations:
Pₙ is a power expressed in diopters which changes one lens to another of said series of lenses of said type with said predetermined increment,
Pₘ is a power expressed in diopters which changes from one lens to another of said series of lenses of said second type with said predetermined increment,
h is the distance from said optical axis expressed in millimeters, and
A (h) is equal to
$${\sum }_{i = 0}^{i = 9} {α}_{2 i} {h}^{2 i}$$
and B (h) is equal to
$${\sum }_{i = 0}^{i = 9} {β}_{2 i} {h}^{2 i} ,$$
for values of h from 0.4 mm to 2.4 mm, the of coefficients α₂ᵢ and β₂ᵢ, for i from 1 to 9, defined by a respective one of the following nine of coefficients SA, SB, SC, MA, MB, MC, LA, LB, LC:

| i | SA | SB | SC |
|---|---|---|---|
| 0 | 1.398800E+00 | 3.093330E+00 | 4.605640E+00 |
| 1 | −2.160020E+00 | −4.751140E+00 | −5.235240E+00 |
| 2 | 1.337720E+00 | 2.913640E+00 | 2.458240E+00 |
| 3 | −4.327890E−01 | −9.378340E−01 | −6.301520E−01 |
| 4 | 8.154230E−02 | 1.764900E−01 | 9.787570E−02 |
| 5 | −9.410290E−03 | −2.038990E−02 | −9.616130E−03 |
| 6 | 6.736380E−04 | 1.462890E−03 | 6.012020E−04 |
| 7 | −2.914960E−05 | −6.347570E−05 | −2.318560E−05 |
| 8 | 6.978470E−07 | 1.520000E−06 | 5.030000E−07 |
| 9 | −7.091930E−09 | −1.550000E−08 | −4.690000E−09 |
| i | MA | MB | MC |
| 0 | 1.799020E+00 | 3.048790E+00 | 4.144890E+00 |
| 1 | −1.823880E+00 | −3.424400E+00 | −4.233760E+00 |
| 2 | 8.133470E−01 | 1.714210E+00 | 1.949870E−00 |
| 3 | −2.057150E−01 | −4.850380E−01 | −5.212190E−01 |
| 4 | 3.222470E−02 | 8.400400E−02 | 8.739800E−02 |
| 5 | −3.231690E−03 | −9.184070E−03 | −9.410210E−03 |
| 6 | 2.075120E−04 | 6.343800E−04 | 6.468110E−04 |
| 7 | −8.241900E−06 | −2.679260E−05 | −2.734250E−05 |
| 8 | 1.842050E−07 | 6.310000E−07 | 6.460000E−07 |
| 9 | −1.770040E−09 | −6.330000E−09 | −6.520000E−09 |
| i | LA | LB | LC |
| 0 | 1.258120E+00 | 2.3409009E+00 | 2.660000E+00 |
| 1 | 2.766510E−01 | −1.6016233E+00 | −3.029760E+00 |
| 2 | −5.863900E−01 | 8.5580090E−01 | 1.837520E+00 |
| 3 | 2.158210E−01 | −4.0855924E−01 | −6.361990E−01 |
| 4 | −3.890640E−02 | 1.2233248E−01 | 1.293960E−01 |
| 5 | 4.063430E−03 | −2.1406740E−02 | −1.595350E−02 |
| 6 | −2.578890E−04 | 2.2148862E−03 | 1.205290E−03 |
| 7 | 9.821560E−06 | −1.3380186E−04 | −5.450000E−05 |
| 8 | −2.065710E−07 | 4.3658573E−06 | 1.350000E−06 |
| 9 | 1.845210E−09 | −5.9468409E−08 | −1.410000E−08 |

in which lists E and the number after it represent a power of 10.

16. The range of lenses claimed in claim 15 wherein said functions A(h) and B(h) are identical and said coefficients α₂ᵢ and β₂ᵢ are taken from the same list.

17. The range of lenses claimed in claim 15 wherein said functions A(h) and B(h) are different and said coefficients α₂ᵢ and β₂ᵢ are taken from two different lists.

18. A method of obtaining a pair of progressive simultaneous vision ophthalmic lenses for correcting the vision of a presbyopic wearer, including the following steps:
a) a step of determining the addition needed for said wearer and the power needed for each eye of said wearer to correct any myopia or hypermetropia,
b) a step of determining which eye of said wearer, referred to as the second eye, has the better tolerance for myopic defocusing, i.e. the blurring introduced by a lens having a positive power,
c) a step of selecting, from range of progressive simultaneous vision ophthalmic lenses including a series of lenses of a first type and a series of lenses of a second type for making up a pair of ophthalmic lenses for correcting the vision of a presbyopic wearer with a first lens for correcting the vision of a first eye of said wearer taken from said series of lenses of said first type and a second lens for correcting the vision of the second eye of said wearer taken from said series of lenses of a second type, in which range of lenses:
each lens from said series of lenses of said first type and from said series of lenses of said second type has a correcting portion whose power, excluding any astigmatism correction, varies as a function of said distance from said optical axis in accordance with a respective progressive profile inscribed in an area between a lower envelope curve and an upper envelope curve, each envelope curve having a respective predetermined polynomial expression, the respective profiles of said lenses of said series of lenses of said first varying with a predetermined power increment, and likewise for said series of lenses of said second type,
for each lens from said series of lenses of said first type the progressive profile in accordance with which its power varies, excluding any astigmatism correction, as a function of said distance from said optical axis is such that said power is greater at a distance of 0.4 mm than at a distance of 2 mm from said optical axis and such that said power at distances from 2 mm to 2.4 mm from said optical axis does not vary by more than 0.5 diopter,
for each lens from said series of lenses of said second type the progressive profile in accordance with which its power varies, excluding any astigmatism correction, as a function of said distance from said optical axis is such that said power is less at a distance of 0.4 mm than at a distance of 2 mm from said optical axis and such that said power at distances from 2 mm to 2.4 mm from said optical axis does not vary by more than 0.5 diopter,
for said first lens, excluding any astigmatism correction, said progressive profile in accordance with which said power varies as a function of said distance from said optical axis is inscribed between a lower envelope curve and an upper envelope curve respectively represented by the following equations:
P1₁(h)=P_{VL1}+A(h)−0.18
P1ᵤ(h)=P1₁(h)+0.36
for said second lens, excluding any astigmatism correction, said progressive profile in accordance with which said power varies as a function of said distance from said optical axis is inscribed between a lower envelope curve and an upper envelope curve respectively represented by the following equations:
P2₁(h)=P_{VL2}+P_{ADD}−B(h)−0.18
P2ᵤ(h)=P2₁(h)+0.36
and, in said equations:
P_{VL1} and P_{VL2} are the powers expressed in diopters, excluding any astigmatism correction, that may be needed to correct near vision for said first eye and for said second eye, respectively,
P_{ADD} is the addition, expressed in diopters, required by the wearer for near vision,
h is the distance from said optical axis expressed in millimeters, and
A (h) is equal to
$${\sum }_{i = 0}^{i = 9} {α}_{2 i} {h}^{{2}_{l}}$$
and B (h) is equal to
$${\sum }_{i = 0}^{i = 9} {β}_{21} {h}^{2 i} ,$$
for values of h from 0.4 mm to 2.4 mm, the series of coefficients α₂ᵢ and β₂ᵢ, for i from 1 to 9, defined by a respective one of the following nine of coefficients SA, SB, SC, MA, MB, MC, LA, LB, LC:

| i | SA | SB | SC |
|---|---|---|---|
| 0 | 1.398800E+00 | 3.093330E+00 | 4.605640E+00 |
| 1 | −2.160020E+00 | −4.751140E+00 | −5.235240E+00 |
| 2 | 1.337720E+00 | 2.913640E+00 | 2.458240E+00 |
| 3 | −4.327890E−01 | −9.378340E−01 | −6.301520E−01 |
| 4 | 8.154230E−02 | 1.764900E−01 | 9.787570E−02 |
| 5 | −9.410290E−03 | −2.038990E−02 | −9.616130E−03 |
| 6 | 6.736380E−04 | 1.462890E−03 | 6.012020E−04 |
| 7 | −2.914960E−05 | −6.347576E−05 | −2.318560E−05 |
| 8 | 6.978470E−07 | 1.520000E−06 | 5.030000E−07 |
| 9 | −7.091930E−09 | −1.550000E−08 | −4.690000E−09 |
| i | MA | MB | MC |
| 0 | 1.799020E+00 | 3.048790E+00 | 4.144890E+00 |
| 1 | −1.823880E+00 | −3.424400E+00 | −4.233760E+00 |
| 2 | 8.133470E−01 | 1.714210E+00 | 1.949870E+00 |
| 3 | −2.057150E−01 | −4.850380E−01 | −5.212190E−01 |
| 4 | 3.222470E−02 | 8.400400E−02 | 8.739800E−02 |
| 5 | −3.231690E−03 | −9.184070E−03 | −9.410210E−03 |
| 6 | 2.075120E−04 | 6.343800E−04 | 6.468110E−04 |
| 7 | −8.241900E−06 | −2.679260E−05 | −2.734250E−05 |
| 8 | 1.842050E−07 | 6.310000E−07 | 6.460000E−07 |
| 9 | −1.770040E−09 | −6.330000E−09 | −6.520000E−09 |
| i | LA | LB | LC |
| 0 | 1.258120E+00 | 2.3409009E+00 | 2.660000E+00 |
| 1 | 2.766510E−01 | −1.6016233E+00 | −3.029760E+00 |
| 2 | −5.863900E−01 | 8.5580090E−01 | 1.837520E+00 |
| 3 | 2.158210E−01 | −4.0855924E−01 | −6.361990E−01 |
| 4 | −3.890640E−02 | 1.2233248E−01 | 1.293960E−01 |
| 5 | 4.063430E−03 | −2.1406740E−02 | −1.595350E−02 |
| 6 | −2.578890E−04 | 2.2148862E−03 | 1.205290E−03 |
| 7 | 9.821560E−06 | −1.3380136E−04 | −5.450000E−05 |
| 8 | −2.065710E−07 | 4.3658573E−06 | 1.350000E−06 |
| 9 | 1.845210E−09 | −5.9468409E−08 | −1.410000E−08 |

in which lists E and the number after it represent a power of 10, a lens from said series of lenses of said first type whose power is equal to the power needed to correct any myopia or hypermetropia of said first eye of said wearer, and
d) a step of selecting from said range of lenses a lens from said series of lenses of said second type whose power is equal to the sum of the power needed to correct any myopia or hypermetropia of said second eye of said wearer and the addition of said wearer.

19. The method claimed in claim 18 further including the following optimization steps:
a step of determining the lens from said series of lenses of said first type whose power for distant vision is the highest possible tolerated by said wearer,
a step of determining the lens from said series of lenses of said second type whose power for near vision is the lowest tolerated by said wearer, and
repeating the preceding two steps alternately, if required, until the best compromise is arrived at.

20. The method claimed in claim 19 wherein said optimization steps are conducted for binocular vision.
